(19) United States
(12) Patent Application Publication
(10) Pub. No.: US 2025/0268759 A1
(43) Pub. Date: Aug. 28, 2025

(54) EYE MASK BODY, EYE MASK, AND PREPARATION METHOD THEREOF

(71) Applicant: CHANGZHOU ZHANLU PLASTIC TECHNOLOGY CO.LTD, Changzhou (CN)

(72) Inventor: Zhen YANG, Changzhou (CN)

(73) Assignee: CHANGZHOU ZHANLU PLASTIC TECHNOLOGY CO.LTD, Changzhou (CN)

(21) Appl. No.: 19/206,178

(22) Filed: May 13, 2025

Related U.S. Application Data
(63) Continuation of application No. PCT/CN2023/131453, filed on Nov. 14, 2023.

(30) Foreign Application Priority Data
Nov. 15, 2022 (CN) 202223040920.5

(51) Int. Cl. A61F 9/04

(52) U.S. Cl. CPC A61F 9/04

(57) ABSTRACT

The present invention relates to an eye mask body, an eye mask, and a preparation method thereof. The eye mask comprises an eye mask body with attached ear straps, where the layers of the eye mask body are heat-pressed at the edge region. The edge pressing marks include an inner pressing line extending along the outer contour of the eye mask body and radial pressing lines radiating outward. The radial pressing lines, located outside the inner pressing line and intersecting the outer contour, can effectively press the edges of the eye mask body to prevent edge exposure as long as the cutting line of the cold-cut eye mask body is within their area. The inner pressing line, while pressing the edges of the eye mask body together with the radial pressing lines, can also simulate sewing lines.

### CROSS-REFERENCE TO RELATED APPLICATIONS

The application claims priority to Chinese patent application No. 202223040920.5, filed on Nov. 15, 2022, the entire contents of which are incorporated herein by reference.

### TECHNICAL FIELD

The present invention relates to an eye mask body, an eye mask, and a preparation method thereof.

### BACKGROUND

In the prior art, the structure of an eye mask typically comprises an eye mask body and ear straps. The layers of the eye mask body are combined into an integral whole through bonding, stitching, ultrasonic welding, or high-frequency heat pressing.

For example, Chinese patent document CN208426304U discloses a disposable instant-cooling specialized cold compress eye mask for ophthalmology, wherein the layers of the eye mask body are combined by bonding, and the ear straps are bonded to both sides of the eye mask body.

Another example is Chinese patent document CN102551954A, which discloses a heat-pressed light-blocking eye mask sheet. The layers of the eye mask body are combined into an integral whole through mold heat pressing, replacing the sewing process.

Bonding the layers of the eye mask body requires adhesives, which is not environmentally friendly and may leave residual odors.

Heat pressing the layers of the eye mask body offers advantages such as convenient production and cost-effectiveness, facilitating continuous production. The eye mask body that has undergone heat pressing needs to be cut off from the raw material through cold cutting method. However, the existing edge heat-pressing designs are unreasonable, leading to edge exposure issues. Moreover, high precision is required for cutting. If misalignment occurs during cold cutting and the cutting line intrudes into the pressed area, edge delamination and exposure of internal materials may occur.

Additionally, eye masks play a significant role in relieving fatigue and aiding sleep during daily life and travel. Light-blocking eye masks primarily function to block light and eliminate interference, creating an environment conducive to sleep, making them suitable for daytime and outdoor use. The light-blocking performance is the most critical indicator for such eye masks.

In the prior art, eye masks often require thick fabrics to achieve light-blocking effects, resulting in poor wearing comfort.

### SUMMARY

The technical problem to be solved by the present invention is: the existing edge heat-pressing designs for eye masks are unreasonable, leading to edge exposure issues.

Another technical problem to be solved by the present invention is: the poor light-blocking effect of existing light-blocking eye masks.

To solve the first technical problem, the present invention adopts the following technical solution: An eye mask comprises an eye mask body and ear straps fixed to the eye mask body. The layers of the eye mask body are pressed together in the edge region. The edge pressing marks include an inner pressing line extending along the outer contour of the eye mask body and radial pressing lines radiating outward. The radial pressing lines are located outside the inner pressing line and intersect with the outer contour of the eye mask body.

Further, the inner pressing line is a single intermittent line, and the radial pressing lines are independent segments.

Further, the inner pressing line is of a penetrating or non-penetrating type, and the radial pressing lines are of a non-penetrating type.

Further, the central part of the eye mask body is provided with a folding pressing line.

Further, the folding pressing line is of a non-penetrating type.

Further, the pressing is achieved through heat pressing.

To solve the second technical problem, the present invention adopts the following technical solution: An eye mask body, specifically a light-blocking eye mask body, comprises an outer light-blocking layer and an inner light-blocking layer stacked together. The colors of the outer and inner light-blocking layers are dark shades.

Further, the light-blocking eye mask body also includes at least one filling layer located between the outer and inner light-blocking layers, outside the outer light-blocking layer, or outside the inner light-blocking layer.

Further, the layers of the light-blocking eye mask body are connected in the edge region through non-sewing methods, such as heat pressing, ultrasonic pressing, or adhesive bonding.

Further, the outer and inner light-blocking layers are non-woven fabrics without warp and weft threads.

Further, the dark shades specifically include black, dark blue, or dark brown.

Further, the filling layer is made of hot-air cotton or sponge.

Further, the non-woven fabric is specifically a spunlace non-woven fabric.

Further, the light-blocking eye mask body is a three-layer structure comprising the outer light-blocking layer, the middle filling layer, and the inner light-blocking layer. The filling layer is located between the outer and inner light-blocking layers, and the layers are pressed together in the edge region through heat pressing.

Further, the outer light-blocking layer is the outermost layer of the eye mask body. The outer surface of the outer light-blocking layer may have decorative patterns, or an additional decorative layer with patterns may be provided outside the outer light-blocking layer, making the decorative layer the outermost layer. An additional comfort layer, such as a cooling layer, warming layer, or skin-friendly soft layer, may be provided outside the inner light-blocking layer.

An eye mask, specifically a light-blocking eye mask, comprises the aforementioned light-blocking eye mask body and ear straps fixed to the eye mask body.

The beneficial effects of the present invention are as follows: By designing radial pressing lines, as long as the cutting line of the eye mask body falls within the area of the radial pressing lines, the radial pressing lines can effectively press the edges of the eye mask body, preventing edge exposure. The inner pressing line, together with the radial pressing lines, can press the edges of the eye mask body while simulating stitching lines.

The dual-layer dark-colored light-blocking layers provide a staggered light-blocking effect, achieving ideal light-blocking performance with lightweight fabrics.

### BRIEF DESCRIPTION OF DRAWINGS

The accompanying drawings are provided to further illustrate the present invention.

FIG. 1 is a front view of the eye mask according to Embodiment 1;

FIG. 2 is a top view of the eye mask according to Embodiment 1;

FIG. 3 is a layer structure diagram of the light-blocking eye mask body according to Embodiment 2;

FIG. 4 is a front view of the light-blocking eye mask according to Embodiment 2;

FIG. 5 is a top view of the light-blocking eye mask according to Embodiment 2;

FIG. 6 is a layer structure diagram of the light-blocking eye mask body according to Embodiment 4;

FIG. 7 is a layer structure diagram of the light-blocking eye mask body according to Embodiment 5;

FIG. 8 is a layer structure diagram of the light-blocking eye mask body according to Embodiment 6.

### REFERENCE NUMERALS

1. Eye mask body
2. Ear straps
3. Inner pressing line
4. Radial pressing lines
5. Folding pressing line
2-1. Outer light-blocking layer
2-2. Inner light-blocking layer
2-3. Filling layer
2-4. Decorative layer
2-5. Comfort layer
2-6. Light-blocking eye mask body
2-7. Ear straps
2-8. Inner pressing line
2-9. Radial pressing lines
2-10. Folding pressing line

### DETAILED DESCRIPTION OF THE EMBODIMENTS

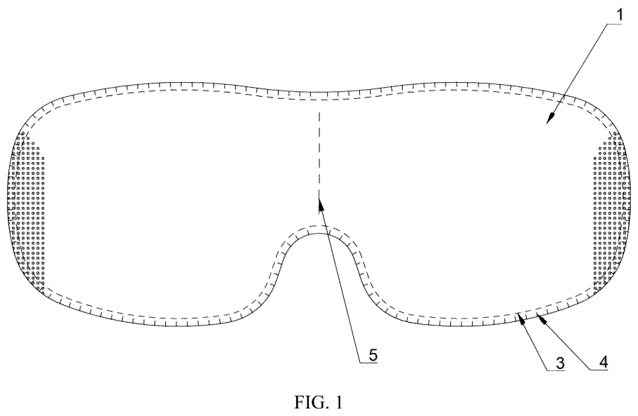

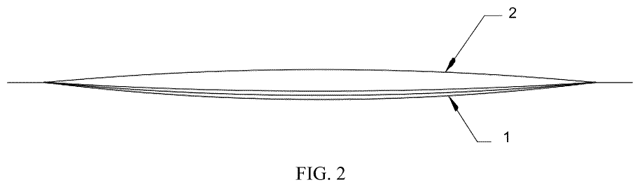

Embodiment 1: As shown in FIGS. 1 and 2, an eye mask comprises an eye mask body 1 and ear straps fixed to the eye mask body 1. The layers of the eye mask body 1 are heat-pressed together in the edge region. The edge pressing marks include an inner pressing line 3 extending along the outer contour of the eye mask body 1 and radial pressing lines 4 radiating outward. The radial pressing lines 4 are located outside the inner pressing line 3 and intersect with the outer contour of the eye mask body 1.

The eye mask body 1 is a three-layer structure comprising a black light-blocking layer, a middle filling layer, and another black light-blocking layer.

The inner pressing line 3 is a single intermittent line, specifically a penetrating or non-penetrating intermittent line, preferably penetrating. This inner pressing line 3 can effectively press the layers of the eye mask body 1 near the edges while simulating stitching lines.

The radial pressing lines 4 are non-penetrating independent segments.

The central part of the eye mask body 1 is provided with a non-penetrating folding pressing line 5.

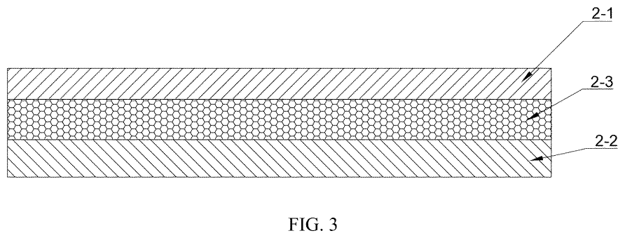

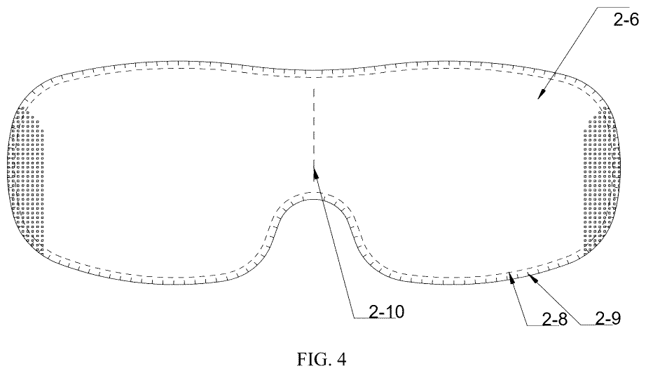

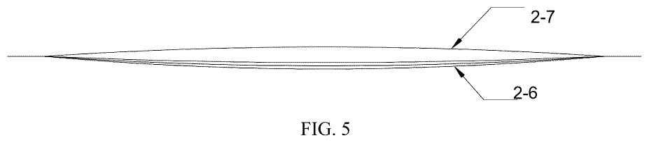

Embodiments 2-6 and related FIGS. 3-8 primarily address the technical problem of poor light-blocking performance in existing light-blocking eye masks.

Embodiment 2: As shown in FIGS. 3, 4, and 5, a light-blocking eye mask body 2-6 comprises an outer light-blocking layer 2-1, a filling layer 2-3, and an inner light-blocking layer 2-2. The outer light-blocking layer 2-1 and inner light-blocking layer 2-2 are stacked, and their colors are dark shades. At least one filling layer 2-3 is located between the outer light-blocking layer 2-1 and the inner light-blocking layer 2-2.

The layers of the light-blocking eye mask body 2-6 are connected in the edge region through non-sewing methods, such as heat pressing, ultrasonic pressing, or adhesive bonding.

Outer light-blocking layer 2-1 and the inner light-blocking layer 2-2 are non-woven fabrics without warp and weft threads.

The dark shades specifically include black, dark blue, or dark brown.

In this embodiment 2, the light-blocking eye mask body 2-6 is a three-layer structure comprising the outer light-blocking layer 2-1, the middle filling layer 2-3, and the inner light-blocking layer 2-2. The filling layer 2-3 is located between the Outer light-blocking layer 2-1 and inner light-blocking layer 2-2, and the layers are pressed together in the edge region through heat pressing. The filling layer 2-3 is a single layer made of hot-air cotton or sponge. Outer light-blocking layer 2-1 and inner light-blocking layer 2-2 are made of non-woven fabric, specifically spunlace non-woven fabric.

The edge pressing marks of the light-blocking eye mask body 2-6 include an inner pressing line 2-8 extending along its outer contour and radial pressing lines 2-9 radiating outward. The radial pressing lines 2-9 are located outside the inner pressing line 2-8 and intersect with the outer contour of the light-blocking eye mask body 2-6.

The central part of the light-blocking eye mask body 2-6 is provided with a folding pressing line 2-10 for easy folding.

An eye mask, specifically a light-blocking eye mask, comprises the light-blocking eye mask body 2-6 in Embodiment 2 and ear straps 2-7. The ear straps 2-7 are fixed to the light-blocking eye mask body 2-6.

In actual production, the outer light-blocking layer 2-1 on the outer side and the inner light-blocking layer 2-2 on the inner side of the light-blocking eye mask body 2-6 are black spunlace fabrics with a weight of 20-100 g, and black spunlace fabrics with a weight of 50 g are preferred.

Outer light-blocking layer 2-1 and inner light-blocking layer 2-2 provide a staggered light-blocking effect, achieving ideal light-blocking performance with lightweight fabrics.

Take Embodiment 2 using a 50-gram black spunlace fabric as Test Object One. Replace the outer light-blocking layer 2-1 of Embodiment 2 with a 100-gram black spunlace fabric and remove the inner light-blocking layer 2-2, which is regarded as the Comparative Test Object. Through actual wearing tests, the light-blocking effect of Embodiment 2 is superior to that of the Comparative Test Object.

Embodiment 3: A light-blocking eye mask body and a light-blocking eye mask are basically the same as those in Embodiment 2. The difference lies in that the outer light-blocking layer 2-1 is located on the outermost layer of the light-blocking eye mask body 2-6, and the outer surface of the outer light-blocking layer 2-1 has a decorative pattern. The decorative pattern can be printed on the outer surface of the outer light-blocking layer 2-1 by means of printing. The layer structure from outside to inside is: outer light-blocking layer 2-1, filling layer 2-3, and inner light-blocking layer 2-2.

Embodiment 4: A light-blocking eye mask body and eye mask, similar to Embodiment 2, except that an additional decorative layer 2-4 with patterns is provided outside the outer light-blocking layer 2-1, making the decorative layer 2-4 the outermost layer.

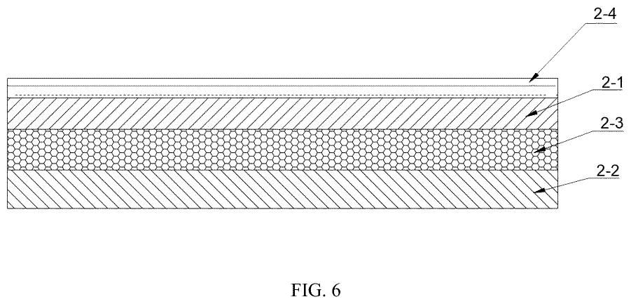

As shown in FIG. 6, the layer structure from outside to inside is: decorative layer 2-4, outer light-blocking layer 2-1, filling layer 2-3, and inner light-blocking layer 2-2.

Embodiment 5: A light-blocking eye mask body and eye mask, similar to Embodiment 4, with an additional comfort layer 2-5 outside the inner light-blocking layer 2-2. The comfort layer 2-5 may be a cooling layer, warming layer, or skin-friendly soft layer.

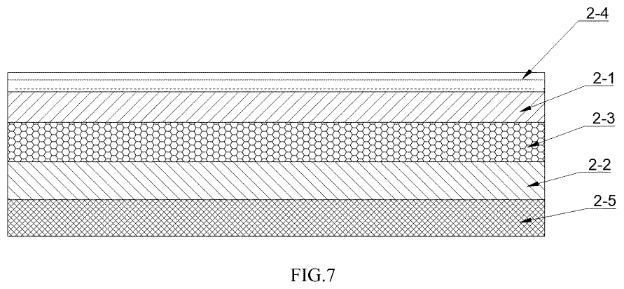

As shown in FIG. 7, the layer structure from outside to inside is: decorative layer 2-4, outer light-blocking layer 2-1, filling layer 2-3, inner light-blocking layer 2-2, and comfort layer 2-5.

Embodiment 6: A light-blocking eye mask body and eye mask, similar to Embodiment 2, except that the filling layer 2-3 is located outside the inner light-blocking layer 2-2.

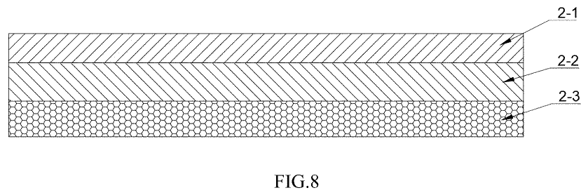

As shown in FIG. 8, the layer structure from outside to inside is: outer light-blocking layer 2-1, inner light-blocking layer 2-2, and filling layer 2-3. An additional comfort layer 2-5 may be provided outside the filling layer 2-3 if needed.

What is claimed is:

1. An eye mask, comprising an eye mask body (1) and ear straps (2) fixed to the eye mask body (1), wherein the layers of the eye mask body (1) are pressed together in the edge region, characterized in that: the edge pressing marks include an inner pressing line (3) extending along the outer contour of the eye mask body (1) and radial pressing lines (4) radiating outward, the radial pressing lines (4) are located outside the inner pressing line (3) and intersect with the outer contour of the eye mask body (1).

2. The eye mask according to claim 1, characterized in that: the inner pressing line (3) is a single intermittent line, and the radial pressing lines (4) are independent segments.

3. The eye mask according to claim 2, characterized in that: the inner pressing line (3) is of a penetrating or non-penetrating type, and the radial pressing lines (4) are of a non-penetrating type.

4. The eye mask according to claim 1, characterized in that: the central part of the eye mask body (1) is provided with a folding pressing line (5).

5. The eye mask according to claim 4, characterized in that: the folding pressing line (5) is of a non-penetrating type.

6. The eye mask according to claim 1, characterized in that: the pressing is achieved through heat pressing.

7. A preparation method for the eye mask according to claim 1, characterized in that: when cutting the eye mask body (1), the cutting line falls within the area of the radial pressing lines (4).

8. An eye mask body, specifically a light-blocking eye mask body (2-6), characterized in that: it comprises an outer light-blocking layer (2-1) and an inner light-blocking layer (2-2) stacked together, and the colors of the outer and inner light-blocking layers are dark shades.

9. The eye mask body according to claim 8, characterized in that: it further comprises at least one filling layer (2-3) located between outer light-blocking layer (2-1) and inner light-blocking layers (2-2);
Alternatively, the said filling layer (2-3) is located on the outer side of the outer light-blocking layer (2-1);
or, the said filling layer (2-3) is located on the outer side of the inner light-blocking layer (2-2).

10. The eye mask body according to claim 8 is characterized in that each layer of the light-blocking eye mask body (2-6) is connected together in the edge area by a non-sewing method, and the non-sewing method specifically includes a hot pressing method, an ultrasonic bonding method or an adhesive bonding method.

11. The eye mask body according to claim 8, characterized in that: the outer light-blocking layer (2-1) and an inner light-blocking layer (2-2) are non-woven fabrics without warp and weft threads.

12. The eye mask body according to claim 8, characterized in that: the dark shades specifically include black, dark blue, or dark brown.

13. The eye mask body according to claim 9, characterized in that: the filling layer (2-3) is made of hot-air cotton or sponge.

14. The eye mask body according to claim 11 is characterized in that the fabric that is not woven with warp and weft threads is specifically a non-woven fabric.

15. The eye mask body according to claim 8, characterized in that: it is a three-layer structure comprising the outer light-blocking layer (2-1), the middle filling layer (2-3), and the inner light-blocking layer (2-2), wherein the filling layer (2-3) is located between the the outer light-blocking layer (2-1) and the inner light-blocking layer (2-2), and the layers are pressed together in the edge region through heat pressing.

16. The eye mask body according to claim 8, characterized in that: the outer light-blocking layer (2-1) is the outermost layer, and its outer surface has decorative patterns; or an additional decorative layer (2-4) with patterns is provided outside the outer light-blocking layer (2-1), making the decorative layer (2-4) the outermost layer; an additional comfort layer (2-5), such as a cooling layer, warming layer, or skin-friendly soft layer, is provided outside the inner light-blocking layer (2-2).

17. An eye mask, specifically a light-blocking eye mask, characterized in that: it comprises the eye mask body according to claim 8 and ear straps (2-7) fixed to the eye mask body.
